(19) United States
(12) Patent Application Publication
(10) Pub. No.: US 2006/0247575 A1
(43) Pub. Date: Nov. 2, 2006

(54) BALLOON CANNULAE

(76) Inventors: Richard Cartledge, Ft. Lauderdale, FL (US); Leonard Y. Lee, New York, NY (US)

Correspondence Address: TROUTMAN SANDERS LLP, 600 PEACHTREE STREET , NE, ATLANTA, GA, 30308, US

(21) Appl. No.: 11/278,276

(22) Filed: Mar. 31, 2006

Related U.S. Application Data
(63) Continuation-in-part of application No. 10/294,366, filed on Nov. 14, 2002.
(60) Provisional application No. 60/344,942, filed on Dec. 21, 2001.

(51) Int. Cl. A61M 29/00

(52) U.S. Cl. 604/102.01

(57) ABSTRACT

A vascular catheter with a circular external diameter, a circular lumen contained within, and an inflatable balloon that overlies a trough in the catheter body such that when the balloon is deflated, the balloon does not protrude beyond the outer circumference of the catheter shaft. The wall of the catheter shaft is thicker along one side than along the remainder of the circumference of the catheter shaft to permit an inflation lumen to be disposed within the thick wall section without increasing the outer diameter of the remaining portion of the circumference. The inflatable balloon is further disposed to maintain the circular lumen of the catheter in the center of flow of the blood vessel in which it is used, maintaining optimal flow. The combination of the circular lumen, circular outer diameter, and recessed balloon trough produces improved hemodynamic flow characteristics within the cannula over existing balloon cannula designs, minimizing flow-related injuries that might cause embolization or vascular dissection.

### CROSS-REFERENCE TO RELATED APPLICATIONS

This application claims priority as a continuation-in-part under 35 U.S.C. § 120 to U.S. patent application Ser. No. 10/294,336 entitled “Balloon Cannulae” and filed Nov. 2, 2002, and to U.S. Provisional Patent Application No. 60/344,942 entitled “Balloon Cannulae” and filed Dec. 21, 2001. The entire contents of these applications are hereby expressly incorporated by reference.

### FIELD OF THE INVENTION

The present invention relates to the fields of cardiovascular and open heart surgery. Specifically, the present invention relates to a novel design for balloon cannulae and to methods of use for such cannulae in surgical procedures involving cardiopulmonary bypass or other high pressure infusion pumps.

### BACKGROUND OF THE INVENTION

The cardiopulmonary bypass machine is one of the most important devices in the field of cardiac surgery. It has enabled cardiac surgeons to safely perform operations for virtually the entire spectrum of acquired and congenital heart diseases. Venous blood is diverted away from the heart and to the cardiopulmonary bypass machine, where it is oxygenated. The oxygenated blood is then returned to the patient via an arterial cannula. Thus the patient's body is properly oxygenated while the heart is stopped. Although it is possible to perform some cardiac procedures without the bypass machine, use of cardiopulmonary bypass remains the cornerstone of modern cardiac surgery.

To shunt venous blood away from the heart and to the cardiopulmonary bypass machine, one or more venous cannulae are used. For many types of coronary bypass surgery, blood is typically shunted out of the body via a single venous cannula. The single venous cannula is inserted through the right atrium and has its tip disposed within the inferior vena cava. Blood enters the cannula through apertures or “fenestrations” in the portion of the cannula shaft that resides within the right atrium, as well as through fenestrations adjacent the tip residing within the inferior vena cava. The single cannula thus diverts the majority of venous return out of the body and into the bypass machine.

During procedures that require actually opening the heart so that the surgeon can access one or more of its internal chambers (i.e., for certain valve and other complex procedures), it is necessary to divert substantially all of the venous blood away from the heart to ensure that blood will not obscure the surgical field. It is also preferable not to have a venous cannula traversing the right atrium, since this not only can obscure the surgeon's view of the internal structures of the heart but also can make manipulation of the heart more difficult. Additionally, it is not desirable to have the fenestrations of the venous cannulae exposed to the ambient air since the bypass circuit should remain filled only with fluid to function efficiently. For these reasons, when surgeons are to perform a procedure that requires total venous diversion, two venous cannulae are used; one to divert blood from the superior vena cava, and one to divert blood from the inferior vena cava. This “bicaval” cannulation shunts substantially all of the venous blood out of the vena cavae to a cardiopulmonary bypass machine, which oxygenates the blood and returns it to the patient via an aortic cannula.

To achieve bicaval cannulation, the surgeon first places a stitch in a circular or “purse-string” configuration at the points of insertion of each cannula, and an incision is made in the tissue central to the respective purse strings. The forward end of one venous cannula is inserted through an insertion point in the anterior wall of the superior vena cava and advanced into the superior vena cava. The second venous cannula is inserted through an insertion point in the inferior lateral aspect of the right atrium and into the inferior vena cava. After the cannulae are placed, the purse strings are cinched around them to create a seal, thus preventing external bleeding or the ingress of air into the cardiopulmonary bypass circuit.

To achieve total venous diversion, clamps or snares must be placed around the superior and inferior vena cavae and cinched down around the distal ends of the cannulae so that no venous blood can pass around and enter the heart. The act of placing these tourniquets involves posterior lateral and posterior medial dissection of the cavae. This surgical maneuver is extremely dangerous and can cause tears in the vena cavae, typically in a posterior location. Additionally, the azygous vein, which joins the superior vena cava in a posterior location, is at risk for injury during dissection of the superior vena cava and snare placement. These tears are extremely difficult to repair because of the limited visual access to their location. Since the cavae are very thin and friable, they are easily injured, with the potential consequence of massive hemorrhage with hemodynamic instability requiring massive transfusions. Such injuries usually necessitate the surgeon to “crash on bypass” before the patient is physiologically ready. If repair can be performed, the repair itself may result in significant narrowing, potentially causing significant morbidity and mortality to the patient. There is also a significant risk of death from such an injury.

Another type of cannula used in a cardiovascular bypass procedure is the arterial cannula. The arterial cannula returns oxygenated blood from the cardiopulmonary bypass machine back to the patient. One such arterial cannula is the arterial balloon cannula, which includes an elongated tubular portion and an expandable balloon at its distal end. A main lumen extends the length of the shaft and has apertures at both its proximal and distal ends. The cannula is inserted through the aortic wall and advanced until the balloon resides within the aortic lumen. When the balloon is inflated, a seal is created between the cannula shaft and the lumen of the aorta to isolate the heart from the cardiopulmonary bypass circuit, thus allowing the surgeon to operate on a non-beating, relatively bloodless organ. With alternate embodiments of arterial cannulae without a balloon, the aorta must be cross-clamped to isolate the heart from the systemic circulation.

When the procedure calls for stopping the heart, cardioplegia solution is administered into the coronary arteries. Cardioplegia solution may be administered either antegrade (through the aortic root and into the coronary arteries) or retrograde (in the reverse direction, from the coronary sinus into the coronary arteries). To administer the cardioplegia solution in retrograde fashion, unidirectional flow must be assured.

Stopping the flow of blood from backing out the administration site can be accomplished by a purse-string placed around the opening to the coronary sinus, with the cardioplegia solution being administered through the retrograde cannula lumen distal to the portion of the cannula shaft that is sealed by the purse-string. But most modern retrograde cardioplegia cannulae avoid the need to place a purse-string in the coronary sinus by providing a balloon affixed to the cannula that is inflated to prevent blood from flowing around the cannula in the wrong direction. Using a balloon retrograde cardioplegia cannula eliminates the time and extra steps it takes to access the coronary sinus and place the purse-string suture.

A conventional balloon retrograde cannula includes an elongated, flexible tubular shaft and an expandable balloon at its distal end. A main lumen extends the length of the shaft and is open at opposite ends of the shaft. The cannula is inserted through the right atrium and advanced until the balloon resides within the coronary sinus. When the balloon at the tip of the cannula is inflated, a seal is created between the cannula shaft and the coronary sinus wall. This seal creates an exclusive communication between the lumen of the cannula and the lumen of the coronary sinus, thus maximizing the efficiency of delivery of cardioplegic solution, blood or other agents into the heart.

An inflated latex balloon in a blood vessel under the pressure of vascular flow may be susceptible to inadvertent displacement, with potential leakage or severe hemorrhage. It would be desirable, therefore, to provide a means of stabilizing such a balloon within the vascular lumen during a surgical procedure. Such a stabilizing means would further have to be retractable or otherwise reducible to allow for easy and atraumatic insertion and removal of the balloon cannula.

Regardless of whether the cannula is a venous cannula, an arterial cannula, or a retrograde cardioplegia cannula, problems are presented with conventional balloon cannulae. The balloon of prior art cannulae is placed on the outside surface of the distal tubular portion, thus creating an acute increase in diameter where the balloon overlies the tubular portion of the cannula. Thus there is a ridge or “step up” where the edge of the balloon is attached to the cannula shaft. This step up can make placement of a conventional balloon cannula problematic, in that it presents a rough location on the surface of the cannula that can cause trauma to the walls of the vessel as the cannula is inserted and withdrawn. In the case of the aortic balloon cannulae, the step up can scrape plaque off the wall of the aorta, which then can embolize in the bloodstream, with adverse clinical consequences. Intimal vascular injuries can also cause dissection injuries, leading to vascular rupture or aneurism formation.

Further, in addition to the main lumen, balloon cannulae must accommodate other accessory lumens for purposes such as inflation of the balloon, cardioplegia infusion pressure monitoring, etc. These conduits typically lie on the exterior surface of the tubular member of the cannula, creating an irregular, non-circular outer cross-sectional profile that can compromise the seal between the vessel/atrial wall and the tubular portion of the cannula. A compromised seal, in turn, can cause blood leakage outside of the anatomic structure the cannula is residing in and potential ingress of air into the circulatory system and/or into the cardiopulmonary bypass circuit, with undesirable clinical consequences. These and other cannulae that include accessory lumens that lie on the internal surface of the cannulae reduce the effective cross-sectional area of the principle lumen and therefore increase resistance and compromise flow of whatever fluid is flowing through the principle lumen.

As an alternative to an eccentric, non-circular outer cross-sectional profile, some existing balloon cannula designs have resorted to maintaining a circular cross-sectional external profile, but making the main lumen in the cannula smaller than the diameter generally employed in such procedures to allow space within a thickened cannular wall to accommodate accessory lumens. This results in disadvantageous flow alterations, with increased resistance, decreased flow capacity, and increased risk of hemolysis from resultant blood cell trauma.

As yet another alternative, some existing balloon cannula designs have resorted to making a cannula with a larger external diameter to accommodate both a standard main lumen and accessory lumens. Such an enlarged cannula requires a surgeon to make a larger hole in a blood vessel for cannula placement, creating increased potential for problems in sealing the cannula, repairing the cannulation site post-procedure, and creating a potential area of dissection or aneurysm formation post-operatively.

Still other existing balloon cannula designs have resorted to making a cannula with a non-circular inner lumen. This is hemodynamically disadvantageous, as a circular lumen results in a more laminar, efficient flow pattern than does a non-circular lumen.

### SUMMARY OF THE INVENTION

The present invention is directed towards a novel design for balloon cannulae to be used when bi-caval cannulation of the heart is indicated, eliminating the need to perform circumferential caval dissection and further reducing the tissue trauma caused by prior art balloon cannulae. Balloon cannulae according to a disclosed embodiment of the present invention comprise inflatable, occlusive balloons adjacent to the cannulae's distal ends. While these cannulae are inserted and positioned by a surgeon in the standard fashion, the need for circumferential dissection of the cavae and tourniquet placement is obviated. After the cannulae are positioned and secured with purse string sutures, the surgeon inflates the occlusive balloons by infusing an inflation medium with a syringe or other means. Once the balloons are inflated, all of the venous return is diverted. The balloons inflate around the distal ends of the cannulae and allow blood to flow through the lumen of the cannulae, but not around the balloons. Use of these cannulae minimizes the chance of caval injury by eliminating the need for circumferential dissection. Additionally, the configuration of the balloon in relation to the cannula is such that the balloon is “flush” with the cannula so that no acute change in diameter exists along the external surface of the cannula, which serves to avoid tissue trauma during insertion and withdrawal into and out of bodily structures.

The present invention addresses several major problems presented by existing designs for balloon cannulae. In various embodiments according to the present invention, the lumens are configured such that a balloon cannula can be inflated without compromising either the flow within the principle lumen of the cannula or the seal between the cannula and the structure within which the cannula lies. Moreover, a disclosed example of a cannula according to the present invention is provided with a trough within the cannula body at its distal end in which the balloon member lies such that when uninflated during insertion and withdrawal, there is a smooth interface between the external cannula wall and the uninflated balloon allowing for smoother, easier, and safer insertion and withdrawal.

Moreover, existing designs for balloon cannulae are unable to provide a truly symmetrical placement of an inflated balloon around a central lumen of standard diameter. The asymmetry which results with conventional balloon inflation is sufficient to displace the lumen from the true center of the endovascular lumen in which the balloon cannula is placed, resulting in unpredictable and suboptimal flow characteristics therethrough. The altered hemodynamics of such flow with an existing balloon cannula increases the likelihood of intimal vascular injury and clot or plaque embolization. Balloon cannulae of the present invention achieve the surprising result of the flow characteristics of a non-balloon cannula by maintaining the preferred laminar flow characteristics of a circular main lumen of consistent diameter, positioned and maintained in or near the center of vascular flow by a balloon originally provided within a recessed trough in the exterior wall of the cannula, with accessory lumens contained within an externally circular cannular wall, allowing for better seal, less vascular trauma, and easier vascular ingress and egress.

In addition, balloon cannulae according to the present invention may be provided with stabilizing elements to anchor the inflated balloon within a vessel lumen during use. Such stabilizing elements further make use of the trough within the cannula body, with the stabilizing elements retracting into said trough during insertion and removal, allowing for smooth and trauma-free entry and egress of the cannula.

Furthermore, balloon cannulae according to the present invention may be provided with a filtration mechanism to collect and prevent embolization of plaques or thrombi that may be caused or displaced by the arteriotomy, venotomy, or cannulation of an artery or vein for placement of the balloon cannulae.

### BRIEF DESCRIPTION OF THE DRAWINGS

FIG. 1 is a side perspective view of a balloon cannula of a disclosed embodiment of the present invention.

FIG. 2 is a side view of the distal portion of the balloon cannula of FIG. 1 with the inflatable balloon illustrated in an uninflated condition.

FIG. 3 is a side view of the distal portion of the balloon cannula of FIG. 1 with the inflatable balloon illustrated in an inflated condition.

FIG. 4 is a side view of the distal portion of the cannula shaft of the balloon cannula of FIG. 1 with balloon removed to show detail.

FIG. 5 is a longitudinal sectional view of the distal portion of the shaft of FIG. 4.

FIG. 6 is a cross-sectional view taken along line 6-6 of FIG. 4.

FIG. 7 is a perspective sectional view taken along line 7-7 of FIG. 4.

FIG. 8 is a longitudinal sectional view of the distal portion of the balloon cannula of FIG. 1 with the balloon in its uninflated condition.

FIG. 9 is a longitudinal sectional view of the distal portion of the balloon cannula of FIG. 1 with the balloon in its inflated condition.

FIG. 10 is a perspective view showing two balloon cannulae of the type illustrated in FIGS. 1-9 positioned within the heart of a patient for performing a cardiac bypass procedure.

FIG. 11 is a side view of an arterial balloon cannula.

FIG. 12 A is a longitudinal view of an exemplary balloon cannula according to the present invention with a portal to allow removable placement of a filtration mesh distal to the cannula during use.

FIG. 12 B is a detail of the distal cannula tip of the arterial cannula of FIG. 12 A.

FIG. 12 C shows an exemplary filtration mesh according to the present invention deployed to cover the great vessel ostia within an aortic arch.

FIG. 12 D shows an external view of an aortic arch and the relationship of an exemplary filtration mesh according to the present invention deployed to cover the great vessel ostia within said arch.

FIG. 12 E is another longitudinal view within an aortic arch showing an exemplary filtration mesh according to the present invention deployed to cover the great vessel ostia therewithin.

FIG. 13 A is a longitudinal view of an exemplary balloon cannula according to the present invention with a portal to permit placement and retrieval of a fixation deployment device therethrough.

FIG. 13 B is a longitudinal view of an exemplary fixation deployment device for use with a balloon cannula according to the present invention.

FIG. 13 C is another longitudinal view of an exemplary fixation deployment device for use with a balloon cannula according to the present invention showing deployment of retainment elements from said device.

FIG. 13 D is another longitudinal view of an exemplary fixation deployment device in use with a balloon cannula according to the present invention showing deployment of retainment elements from said device.

FIG. 14 A is a longitudinal view of the distal tip of an exemplary balloon cannula according to the present invention, with a portal and a retention lumen to allow placement and removal of a retention wire and retention element therethough.

FIG. 14 B is a longitudinal view of the exemplary balloon cannula of FIG. 14 A with deployment of a retention wire and retention element therethough, but with the balloon of said cannula deflated.

FIG. 14 C is a longitudinal view of the exemplary balloon cannula of FIG. 14 A with deployment of a retention wire and retention element therethough, but with the balloon of said cannula inflated.

FIG. 15 A is a longitudinal section of an uninflated magnetic balloon cannula according to the present invention positioned within a blood vessel lumen.

FIG. 15 B is a cross section of an uninflated magnetic balloon cannula according to the present invention positioned within a blood vessel lumen.

FIG. 15 C is a longitudinal section of an inflated magnetic balloon cannula according to the present invention positioned within a blood vessel lumen, showing displacement of an intralumenal magnet against the luminal wall of the vessel.

FIG. 15 D is a cross section of an inflated magnetic balloon cannula according to the present invention positioned within a blood vessel lumen, showing displacement of an intralumenal magnet against the luminal wall of the vessel.

FIG. 15 E is a longitudinal section of an uninflated magnetic balloon cannula with multiple intralumenal magnets according to the present invention positioned within a blood vessel lumen.

FIG. 15 F is a cross section of an uninflated magnetic balloon cannula with multiple intralumenal magnets according to the present invention positioned within a blood vessel lumen.

FIG. 15 G is a longitudinal section of an inflated magnetic balloon cannula with multiple intralumenal magnets according to the present invention positioned within a blood vessel lumen, showing displacement of the intralumenal magnets against the luminal wall of the vessel.

FIG. 15 H is a cross section of an inflated magnetic balloon cannula with multiple intralumenal magnets according to the present invention positioned within a blood vessel lumen, showing displacement of the intralumenal magnets against the luminal wall of the vessel.

FIG. 16 A shows in cross section an exemplary extravascular magnetic collar according to the present invention with a hinged connection.

FIG. 16 B shows in cross section an exemplary extravascular magnetic collar according to the present invention with a hinged connection applied around a blood vessel containing a single intralumenal magnet.

FIG. 16 C shows in cross section an exemplary extravascular magnetic collar according to the present invention with a hinged connection applied around a blood vessel containing a plurality of intralumenal magnets.

FIG. 17 A is a cross sectional view of a portion of the wall of an inflatable balloon on an exemplary cannula according to the present invention in which an intralumenal magnet is located within the structure of said wall, but substantially flush with the outer surface of said balloon.

FIG. 17 B is a cross sectional view of a portion of the wall of an inflatable balloon on an exemplary cannula according to the present invention in which an intralumenal magnet is located substantially centrally within the structure of said balloon wall.

FIG. 17 C is a cross sectional view of a portion of the wall of an inflatable balloon on an exemplary cannula according to the present invention in which an intralumenal magnet is located substantially on the exterior surface of said balloon wall.

FIG. 18 A is a side view of the distal portion of an exemplary cannula according to the present invention in which the balloon is provided with transverse retention elements and is in an uninflated condition.

FIG. 18 B is a side view of the distal portion of an exemplary cannula according to the present invention in which the balloon is provided with transverse retention elements and is in an inflated condition.

FIG. 19 A is a side perspective view of an arterial balloon cannula of a disclosed embodiment of the present invention with the balloon in an uninflated condition.

FIG. 19 B is a side perspective view of an arterial balloon cannula of a disclosed embodiment of the present invention with the balloon in an inflated condition.

### DETAILED DESCRIPTION OF DISCLOSED EMBODIMENTS

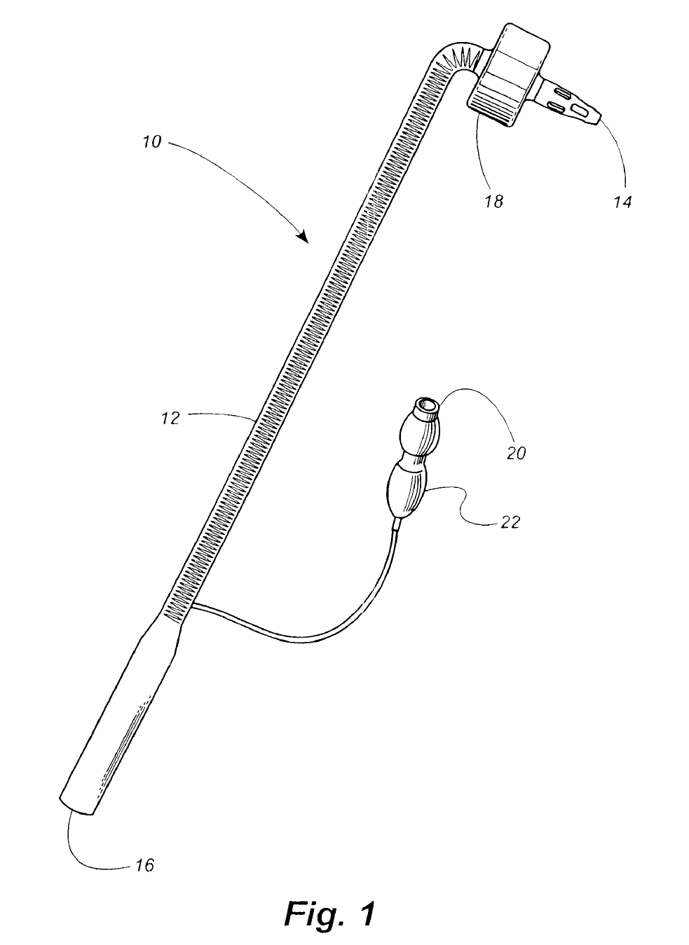

Referring now in more detail to the drawings, in which like numerals indicate like elements throughout the several views, FIG. 1 illustrates a balloon cannula 10 according to a disclosed embodiment of the present invention. The balloon cannula 10 includes an elongated, tubular cannula shaft 12 having a distal end 14 and a proximal end 16. In the disclosed embodiment, the cannula body 12 is constructed of a pliable material such as, but not limited to, natural or synthetic rubbers, elastomers, polyisoprenes, polyurethanes, vinyl plastisols, acrylic polyesters, polyvinylpyrrolidone-polyurethane interpolymers, butadiene rubbers, styrene-butadiene rubbers, rubber lattices, and other polymers or materials with similar resilience and pliability qualities. An inflatable member or balloon 18 is located adjacent the distal end 14 of the cannula shaft 12. An inflation valve 20 adapted to accommodate a syringe or other suitable apparatus branches off from the cannula shaft 12 at a location adjacent the proximal end 16 of the cannula shaft. A syringe can be coupled to the inflation valve 20 to inflate or to deflate the balloon 18 by infusing liquids or gases into the inflatable member through an inflation lumen. In the disclosed embodiment, a pilot balloon 22 is provided in-line between the inflation valve 20 and the balloon 18. The pilot balloon 22 is inflatable and is designed to provide a user an indication of the degree of inflation of the balloon 18.

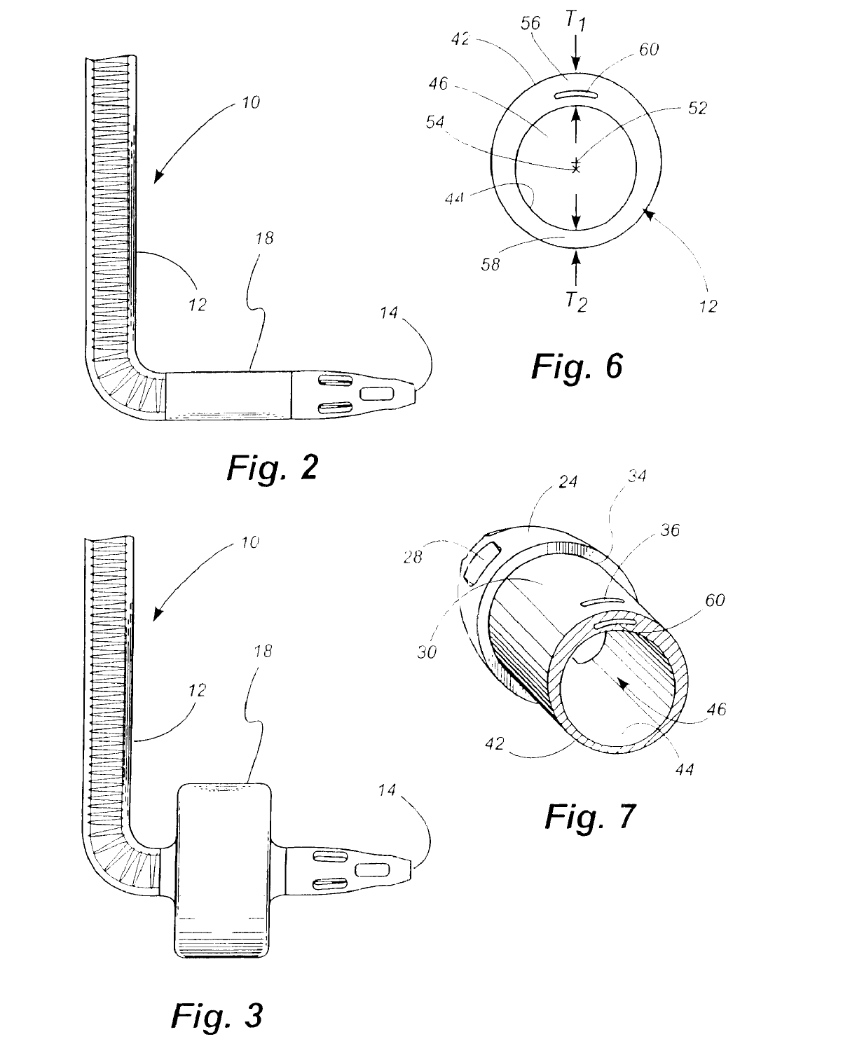

FIG. 2 illustrates the distal portion of the balloon cannula 10 with the balloon 18 in its uninflated condition. FIG. 3 illustrates the distal portion of the balloon cannula 10 with the balloon 18 in its inflated condition.

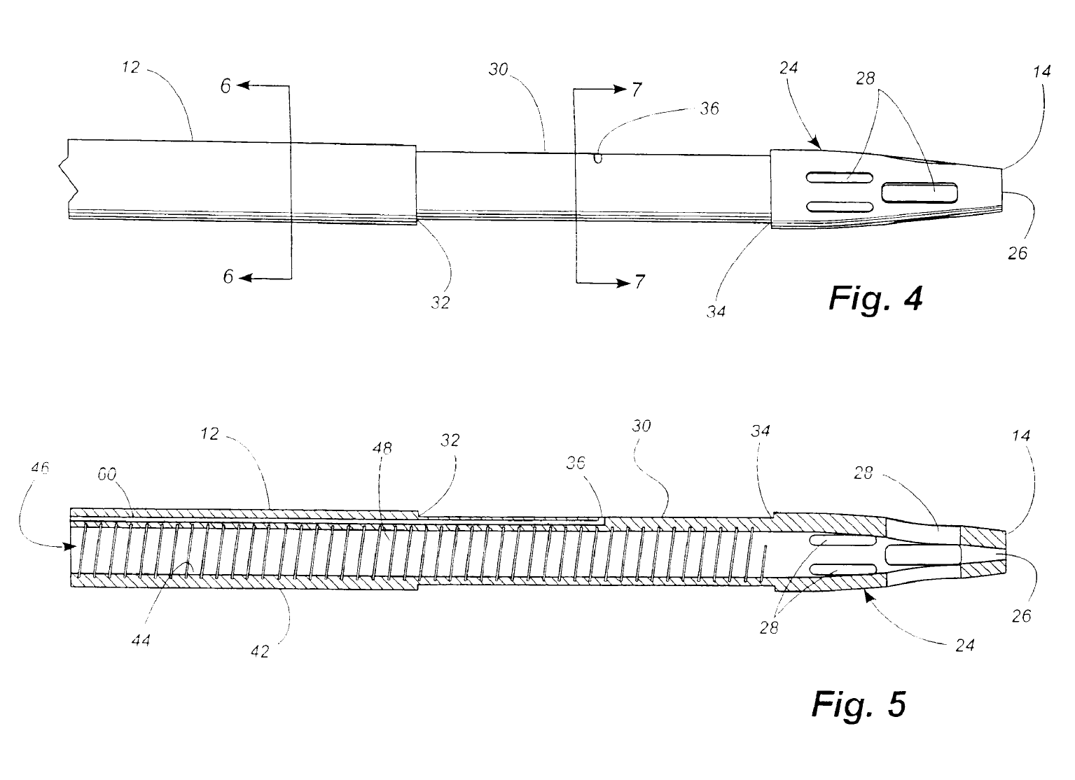

Reference is now made to FIG. 4 of the drawings, in which the distal portion of the cannula shaft 12 is illustrated. A cannula tip 24 located at the forward end of the shaft 12 is tapered to facilitate introduction. An opening 26 is formed in the forward end of the cannula tip 24. In addition, lateral openings 28 are formed in the sides of the cannula tip 24. In alternate embodiments, the cannula tip can be rounded instead of tapered, the end of the distal tip 14 may be closed instead of open, or it may be provided with one or more central apertures 26. The lateral apertures 28 may be provided in addition to or in place of the central aperture 26 or may be eliminated entirely.

Just rearward of the cannula tip 24, a portion of the shaft 12 has a reduced diameter so as to form a circumferential trough 30. The trough 30 has a proximal edge 32 and a distal edge 34. An inflation aperture 36 is formed in the wall of the trough portion 30.

Referring now to FIG. 5, the cannula shaft 12 has an exterior surface 42 and an interior surface 44. The cannula shaft 12 defines a main lumen 46 that extends the length of the shaft. The axial tip opening 26 and the lateral openings 28 place the cannula lumen 46 in fluid communication with the ambient surrounding the cannula tip 24.

To provide structural reinforcement for the main lumen 46, a coil 48 may extend along some or all of the length of the cannula shaft 12. The coil 48 is preferably embedded within the wall of the cannula shaft 12 but in alternate embodiments may wrap around the exterior of the cannula wall 18 or may be positioned within the cannula lumen 46. The coil 48 may be constructed of a wire of stainless steel or other suitable metal or of plastics sufficiently stiff to increase cannula strength and to prevent undesirable kinking that might adversely affect flow within the balloon cannula during use.

As can be seen in FIG. 6, the cannula shaft 12 has a longitudinal axis 52, and the main lumen 46 of the cannula shaft has a longitudinal axis 54. The longitudinal axis 54 of the main lumen 46 is offset from the longitudinal axis 52 of the cannula shaft 12 such that the main lumen is eccentric with respect to the cannula shaft. As a consequence, a portion 56 of the wall on one side of the main lumen 46 has a thickness T₁ that is greater than the thickness T₂ of the wall portion 58 on the opposite side of the main lumen. As shown in FIG. 7, an inflation lumen 60 is formed in the thicker portion 56 of the wall and communicates with the inflation aperture 36 in the trough portion 30 of the cannula shaft 12. The asymmetrical configuration permits the inflation lumen to be incorporated into the wall of the cannula shaft 12 to eliminate the protrusion of a separate inflation tube on the periphery of the cannula body. At the same time, since only the portion 56 of the wall defining the inflation lumen 60 is thickened, the overall circumference of the cannula shaft 12 is minimized.

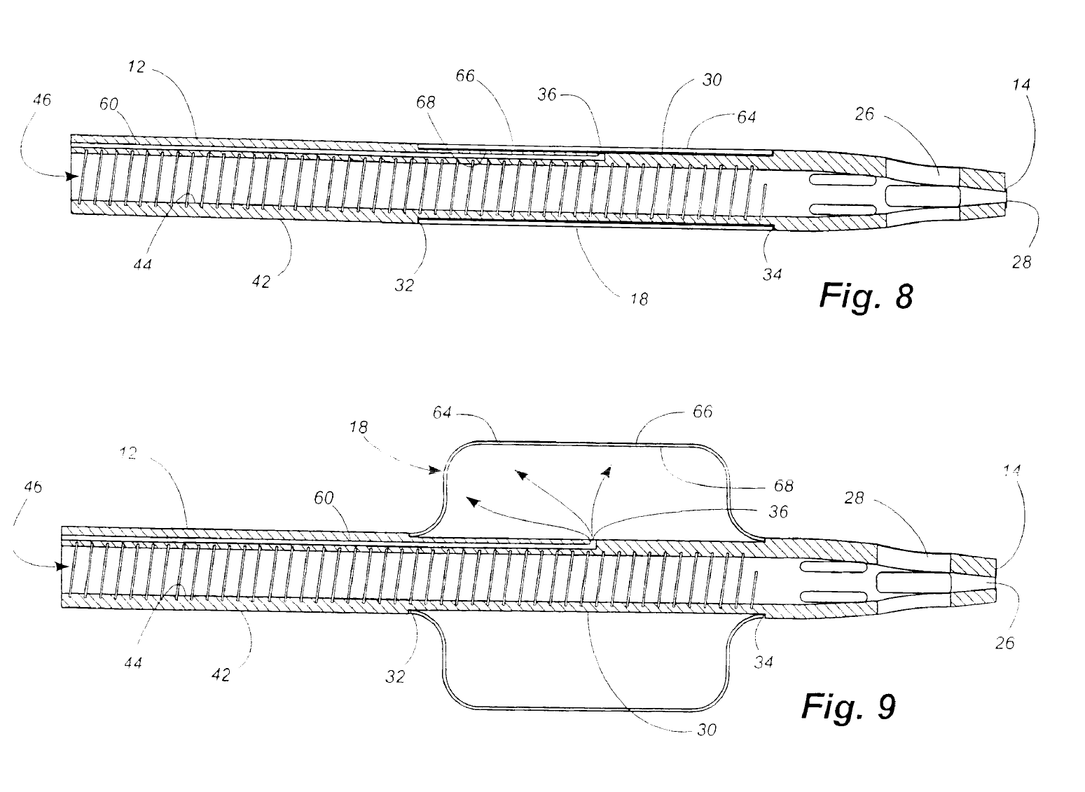

Referring now to FIG. 8, the balloon 18 is shown fastened to the cannula shaft 12 and in its uninflated condition. The balloon 18 includes a wall 64 of a fluid-impermeable material. The wall 64 of the balloon 18 has an exterior surface 66 and an interior surface 68. The balloon 18 overlies the trough portion 30 of the cannula shaft 12 and the inflation aperture 36 located thereon. The balloon 18 is secured at its rearward and forward ends to the proximal and distal end portions 32, 34 of the trough 30. In its uninflated condition, the interior surface 68 of the wall 64 of the balloon 18 is imposed against the trough portion 30 of the cannula shaft 12, and the exterior surface of the balloon is flush with the exterior surface 42 of the main portion of the cannula shaft 12. Stated differently, the reduction in diameter of the trough portion 30 is equal to the thickness of the wall 64 of the balloon 18 in its uninflated state. By recessing the balloon 18 into the trough 30 in this manner, there is no portion of enlarged circumference that might cause trauma to a bodily structure upon insertion and again upon withdrawal of the cannula 10.

FIG. 9 depicts the balloon 18 in its inflated state. An infusion medium such as a saline solution is infused by a syringe or other suitable means into the inflation port 20 (FIG. 1), through the inflation lumen 60, and out the inflation aperture 36 beneath the surface of the balloon. In response, the balloon 18 expands radially outward.

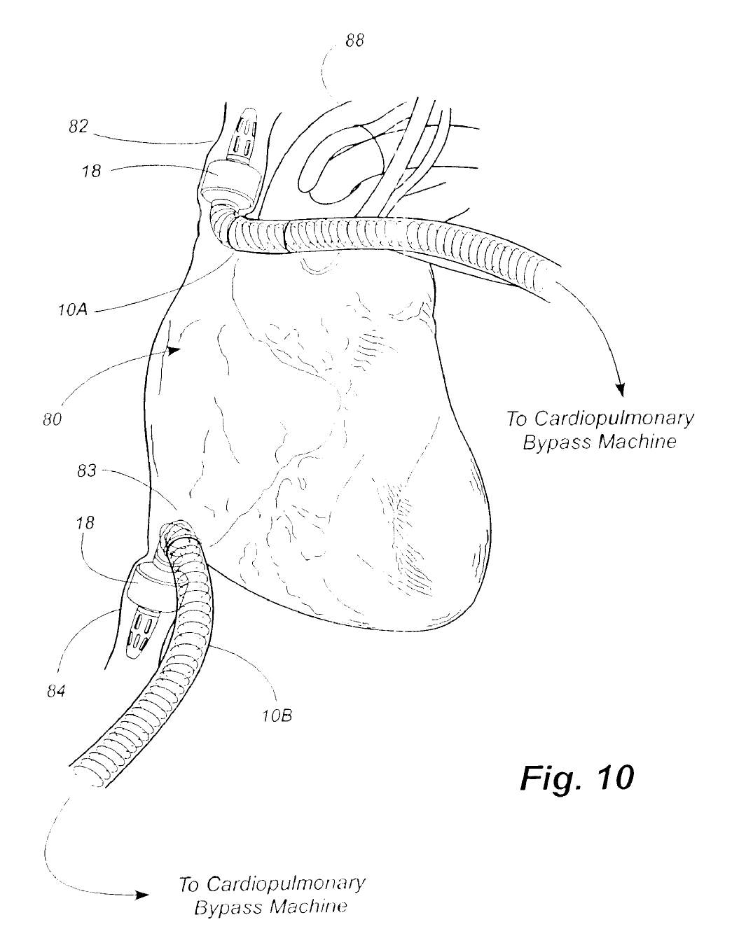

FIG. 10 illustrates the use of a pair of balloon cannulae 10 to perform a bicaval cannulation for the purpose of diverting venous blood away from a heart 80 and to a cardiopulmonary bypass machine. First, a stitch is placed in a circular or “purse-string” configuration at a first insertion point on the anterior wall of the superior vena cava 82. Another purse-string stitch is placed at a second insertion point in the inferior lateral aspect of the right atrium 83. An incision is made in the tissue central to the respective purse strings. A first balloon cannula 10A is then advanced through the circular stitch at the first insertion point and into the superior vena cava 82. A second balloon cannula 10B is advanced through the circular stitch at the second insertion point and into the inferior vena cava 84. The purse strings are then cinched around the balloon cannulae 10A, 10B to create a seal, thus preventing external bleeding or the ingress of air into the cardiopulmonary bypass circuit.

At this point sufficient blood is shunted to the cardiopulmonary bypass machine for a bypass procedure to begin. Return flow of oxygenated blood from the bypass machine is directed through an arterial cannula (not identified in FIG. 10) to the aorta 88. If it is necessary to divert the complete flow of blood, the balloons 18 of each of the cannulae 10A, 10B are inflated, forming a fluid-tight seal between the cannula shaft and the wall of the respective vena cavae 82, 84. Now all of the venous blood flow is diverted to the bypass machine, creating a clear field of view for the surgeon to operate.

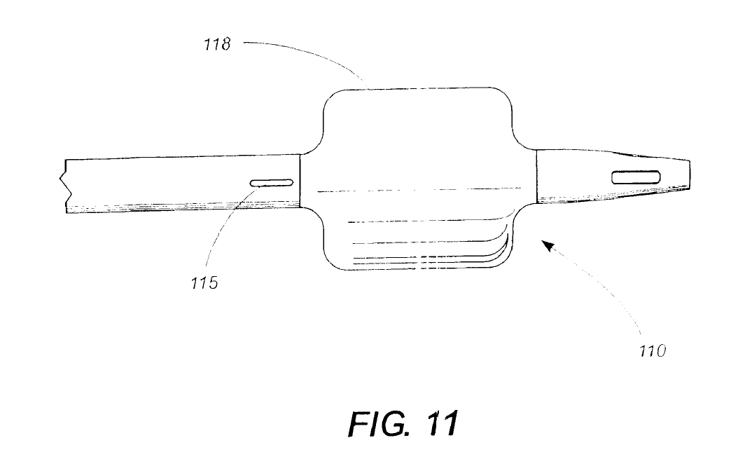

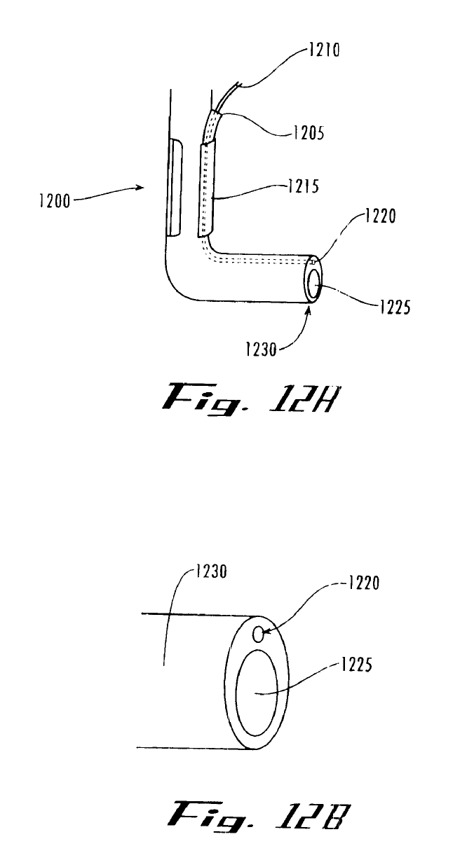

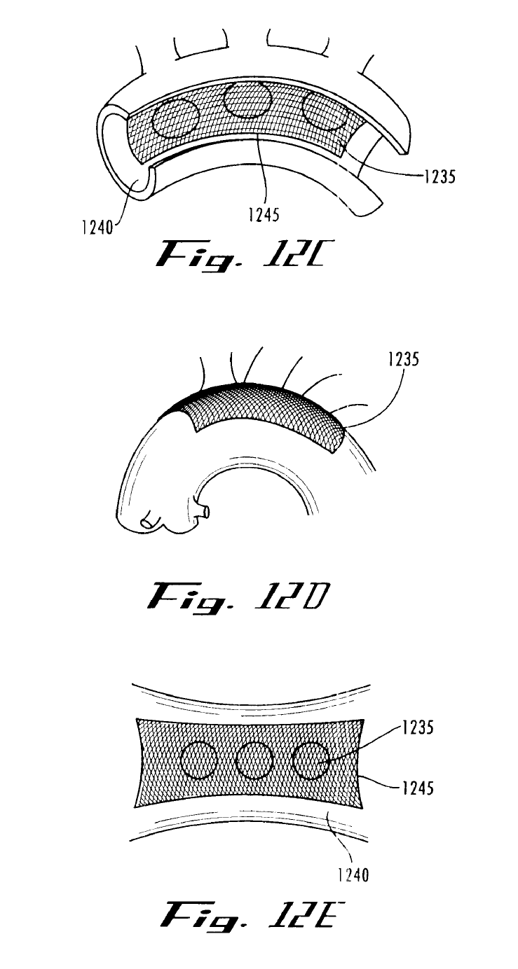

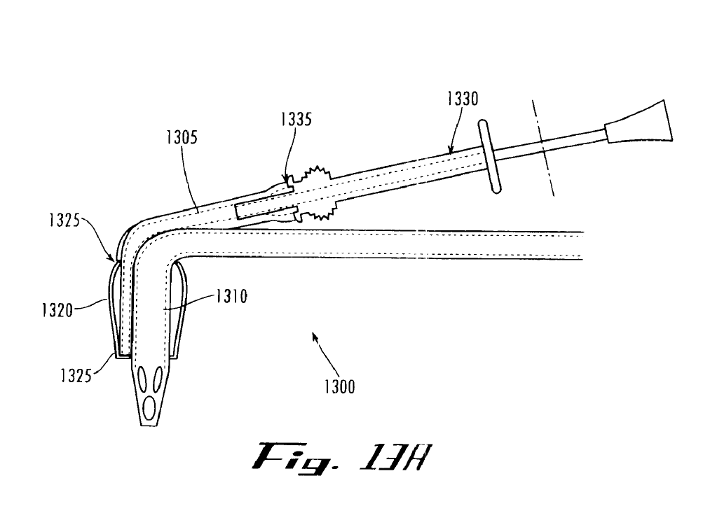

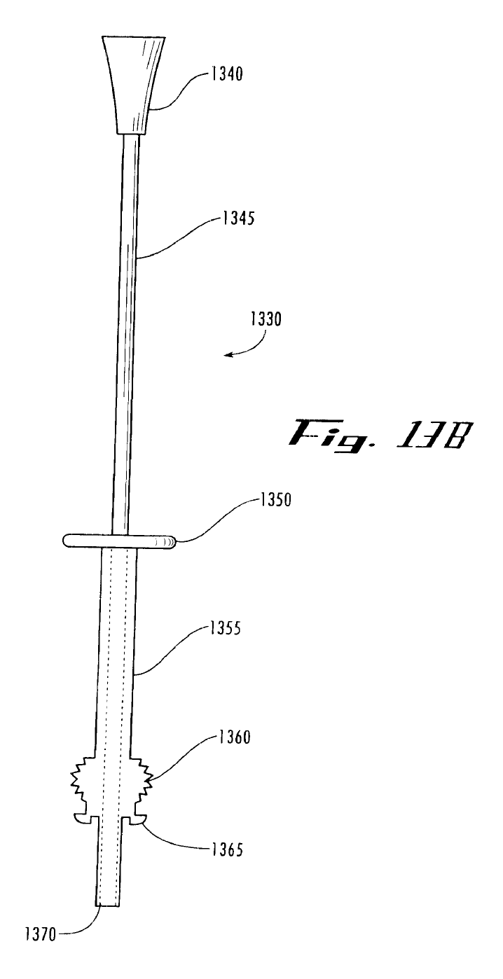

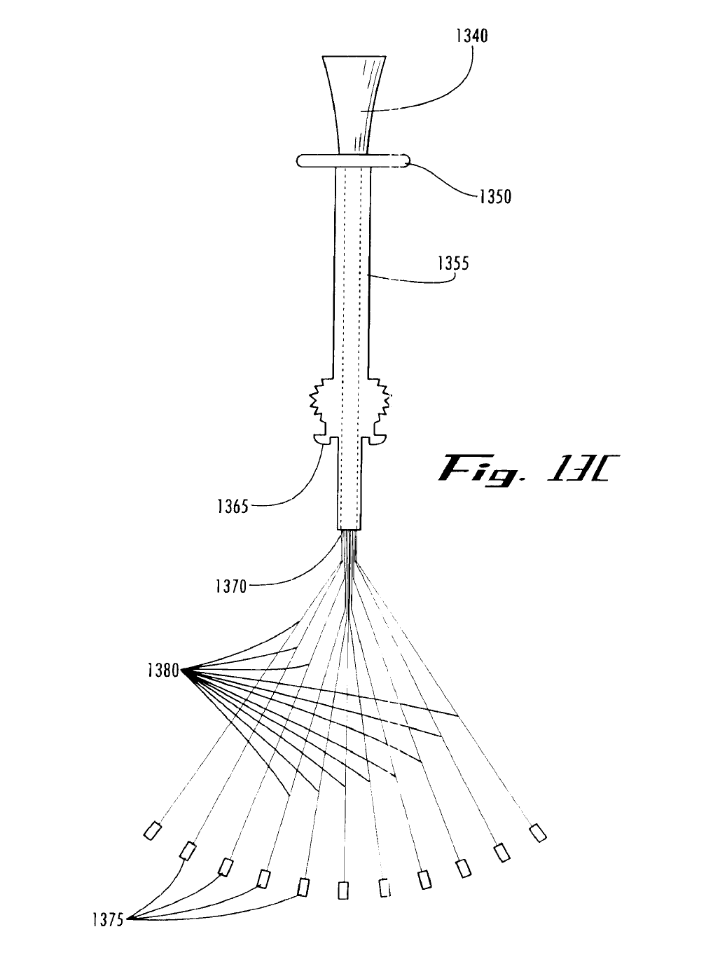

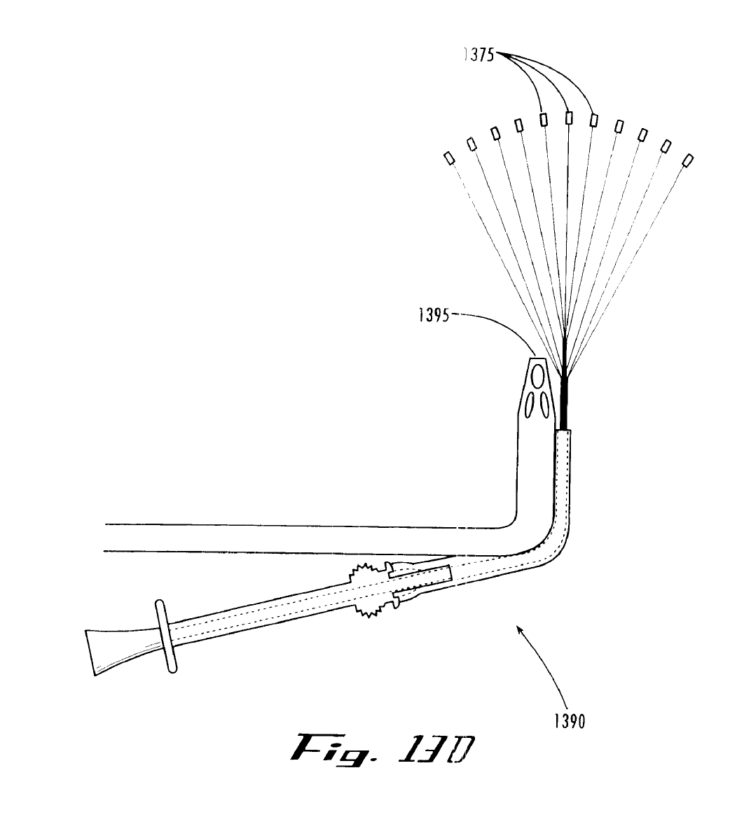

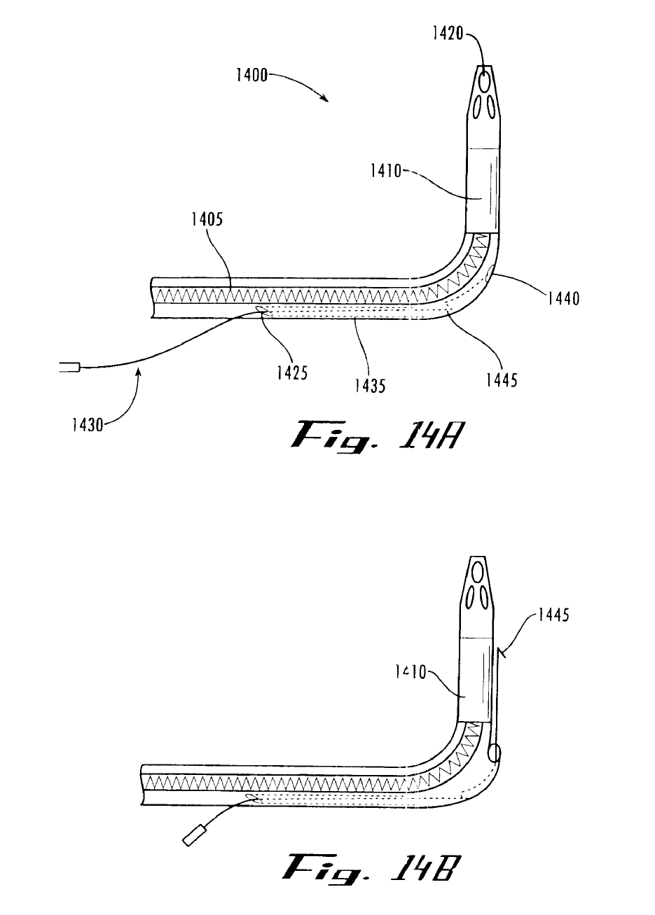

FIG. 11 illustrates a cannula 110 that is suitable for use as an arterial balloon cannula. In addition to a main lumen and an inflation lumen, the cannula 110 includes an accessory lumen that terminates in a port 115 proximal to the balloon 118. The cannula 110 is inserted through the aortic wall and advanced until the balloon resides within the aortic lumen. When the balloon 118 is inflated, a seal is created between the cannula shaft and the lumen of the aorta to isolate the heart from the cardiopulmonary bypass circuit, thus allowing the surgeon to operate on a non-beating, relatively bloodless organ. Cardioplegia can then be administered through the accessory lumen and out of the port 115.

In various embodiments according to the present invention, the thickness of the cannula wall may increase through some or all of its length, such that there may be a cannula taper in which the external diameter of the cannula shaft 12 increases towards the proximal end 16 of the cannula 10. In all cases, however, the diameter of the cannula lumen 46 as defined by the circumference of the internal cannula wall 20 remains constant throughout the length of the device 10. The constant diameter of the cannula lumen 46 serves to maintain an even flow therethrough.

The flow dynamics of an exemplary venous balloon cannula according to the present invention are represented in Table 1. This flow data is consistent with the dynamic results of a conventional cannula with a round inner luminal cross-section, but are surprising for a balloon cannula in that most balloon cannulae are unable to maintain a perfectly circular cross-sectional lumen, and are further unable to maintain the center of their lumens in the exact center of the vessel's anatomic lumen, where flow dynamics are maximized. Thus, the combination of the offset, but circular cannula lumen and the recessed trough balloon that allows exact placement within the vessel's anatomic lumen works together to yield a cannula with the best functional advantages of a balloon device with the best functional advantages of a circular cannula. The recessed trough design of balloon cannulae according to the present invention further allows for inclusion of retractable retention means that serve to stabilize the desired endovascular positioning of the balloon cannula during use, prevent inadvertent displacement of the same during use, and yet allows easy removal of the balloon cannula after use. The advantages of an offset circular cannula with the recessed trough balloon are further increased by utilization of additional offset lumens within the distal tip of the cannula that may be provided to allow introduction of devices to retractably stabilize the deployment of an inflated balloon cannula in situ and/or devices provided to trap and collect endovascular plaques or thrombi that may be displaced by or caused by the arteriotomy, venotomy, or endovascular introduction of the balloon cannula.

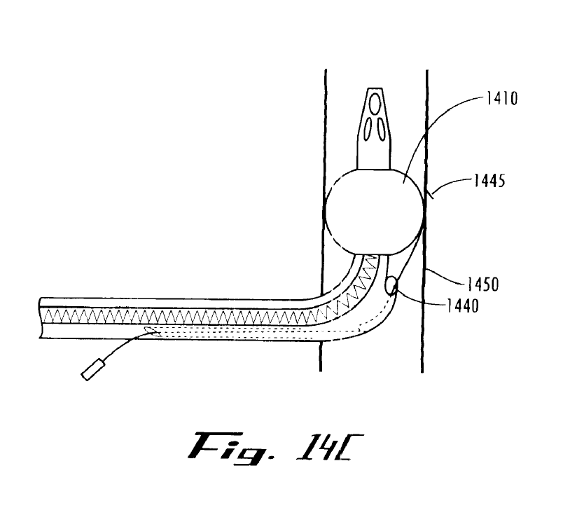

FIG. 12 A shows the distal aspect of a balloon cannula 1200 according to the present invention wherein an access port 1205 is provided proximal to the recessed trough/balloon 1215 that is in direct communication with an egress port 1220 which is located at the distal tip 1230 of the cannula 1200, with the egress port 1220 located parallel to the cannula lumen 1225, but offset from the central axis of the cannula tip 1230. The cannula tip 1230 is further detailed in FIG. 12B.

The access port 1205 in such an embodiment according to the present invention receives an introduction device 1210 which is inserted to extend through and past the egress port 1220 and manipulated to deploy a filtration mesh 1235 and a retractable mesh frame 1245 as shown in FIG. 12 C-E. In this example, the filtration mesh 1235 is deployed by expansion of the retractable mesh frame 1245 so as to cover the orifices of the innominate, left subclavian, and left common carotid vessels present in the aortic arch of a human heart. Such a filtration mesh may constructed of a snag-free material with mesh openings sized to prevent particulate matter from passing therethrough, but capable of permitting blood flow therethrough. Such a filtration mesh 1235 may be floppy to allow its partial invagination into the orifice of connecting blood vessels under the pressure of blood flow therein. Such a filtration mesh 1235 may be constructed of metal, plastics, or monofilament or woven polymers, or any combination thereof. Such a mesh frame 1245 may be collapsed to allow removal of the filtration mesh 1235 and removal of the introduction device from the cannula 1200 through the access port 1205 at the completion of the surgical procedure.

Alternately, as shown in FIGS. 13 A-D, a portal 1305 is located parallel and adjacent to a cannula lumen 1310, both contained within a balloon cannula 1300 with an inflatable balloon 1320 recessed within a trough 1325, such that, when the balloon 1320 is not inflated, said balloon 1320 is substantially flushly housed within said trough 1325. Said portal 1305 is sized and provided to receive a fixation deployment device 1330 at an access port 1335. In an exemplary embodiment as detailed in FIG. 13 B, the fixation deployment device 1330 comprises a plunger tip 1340, a slidable shaft 1345, a plunger stop 1350, a distal tube 1355, a containment section 1360, a portal connector 1365, and a distal tip 1370. When the plunger tip 1340 is depressed towards the plunger stop 1350, one or more extension members 1380, as shown in FIG. 13 C, is displaced from the containment section 1360, with each extension member 1380 terminating in a retainment element 1375. Said retainment elements 1375 may be provided with barbs, hooks, a frictional surface, or other means of engaging adjacent tissue and serving to retain the placement and positioning of the device 1330. In various embodiments, the retainment elements 1375 may be deployed in front of, at the same level as, or behind the distal tip 1370. FIG. 13 D shows deployment of such a fixation deployment device 1330 within a balloon cannula 1390 according to the present invention, with the retainment members 1375 extending distally beyond the cannula tip 1395 to stabilize and maintain positioning of the entire cannula 1390. In various embodiments according to the present invention, one or more extension members 1380, as shown in FIG. 13 C, may be deployed attached to a filtration mesh (not shown) to collect displaced plaques or other potentially embolic debris during cannula use. In such embodiments, the filtration mesh might be deployed in an umbrella-like manner, and then collapsed when cannula removal is desired.

FIG. 14 A-C details an alternate embodiment according to the present invention, with a balloon cannula 1400 comprising an offset circular cannula lumen 1405 ending at a distal cannula tip 1420. Proximal to the distal cannula tip 1420, an integral inflatable balloon 1410 is substantially flushly housed in a trough (not shown) in the cannula 1400 when said balloon 1410 is deflated. Parallel but offset from said cannula lumen 1405, a retention lumen 1435 extends from an access port 1425 to an egress port 1440. A retention wire 1430 is received by the access port 1425 and passes through the retention lumen 1435 to exit at the egress port 1440. The retention wire 1430 ends with a retention element 1445 which may be a hook, barb, or frictional surface. In use, as shown in FIG. 14 B, the retention element 1445 is extended until it is adjacent to the deflated balloon 1410. When the balloon 1410 is inflated, the retention element 1445 is pressed into contact with a blood vessel wall 1450, engaging said blood vessel wall 1450 and holding the balloon cannula 1400 in position during use. When the procedure is completed and the balloon is deflated, the retention element 1445 is advance slightly, disengaging from the blood vessel wall 1450 so that the retention element 1445 may be withdrawn through the egress port 1440.

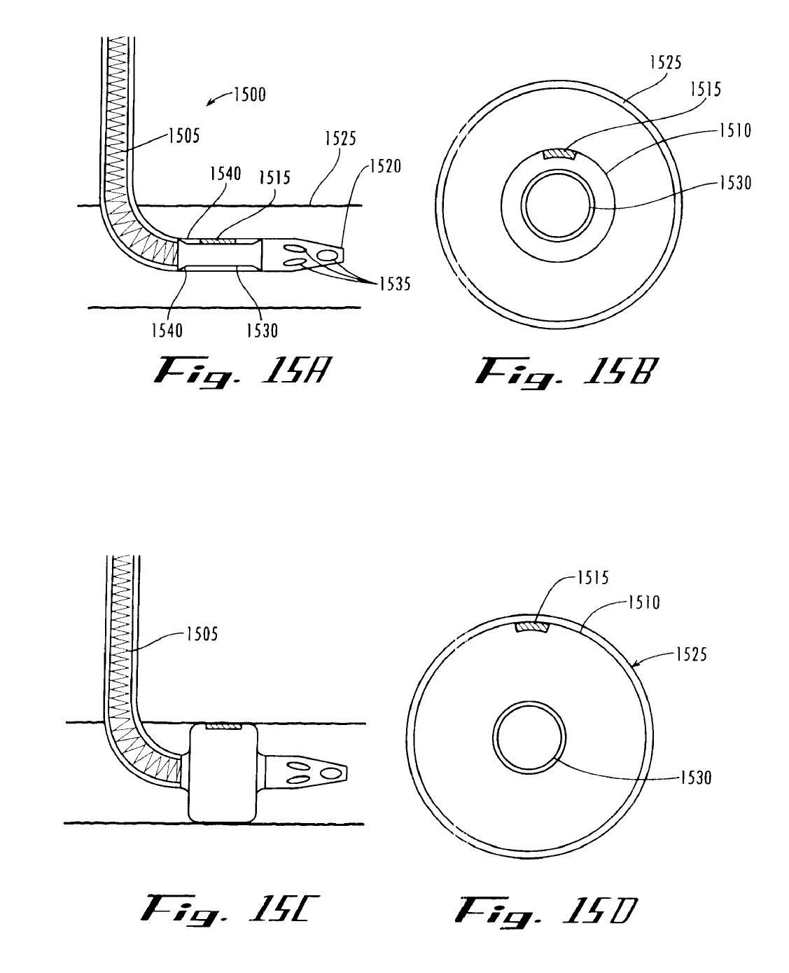

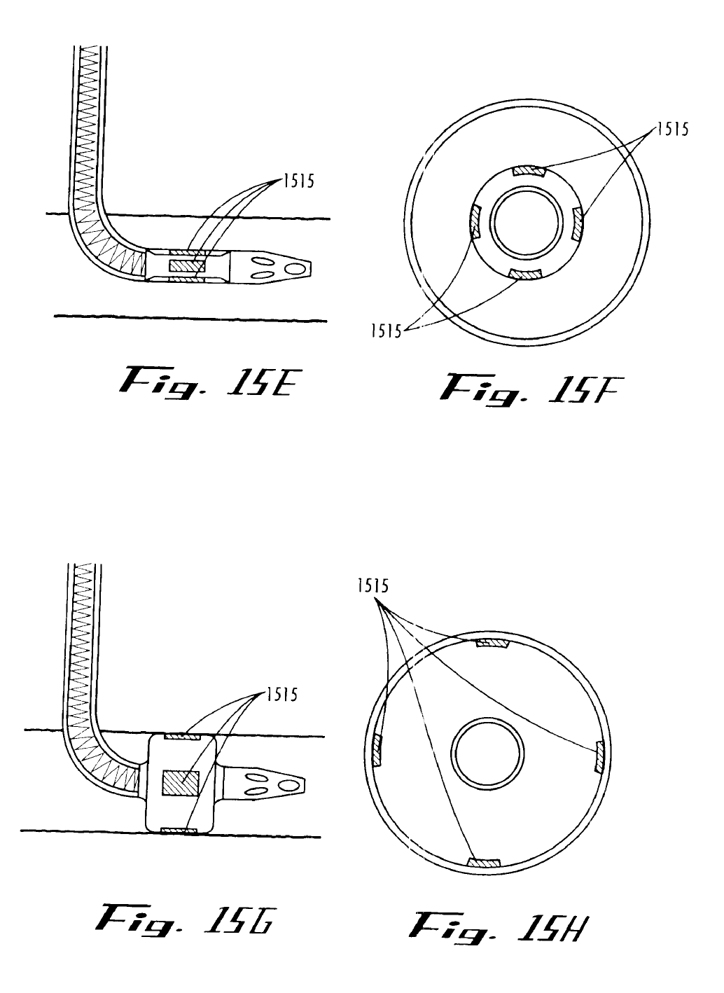

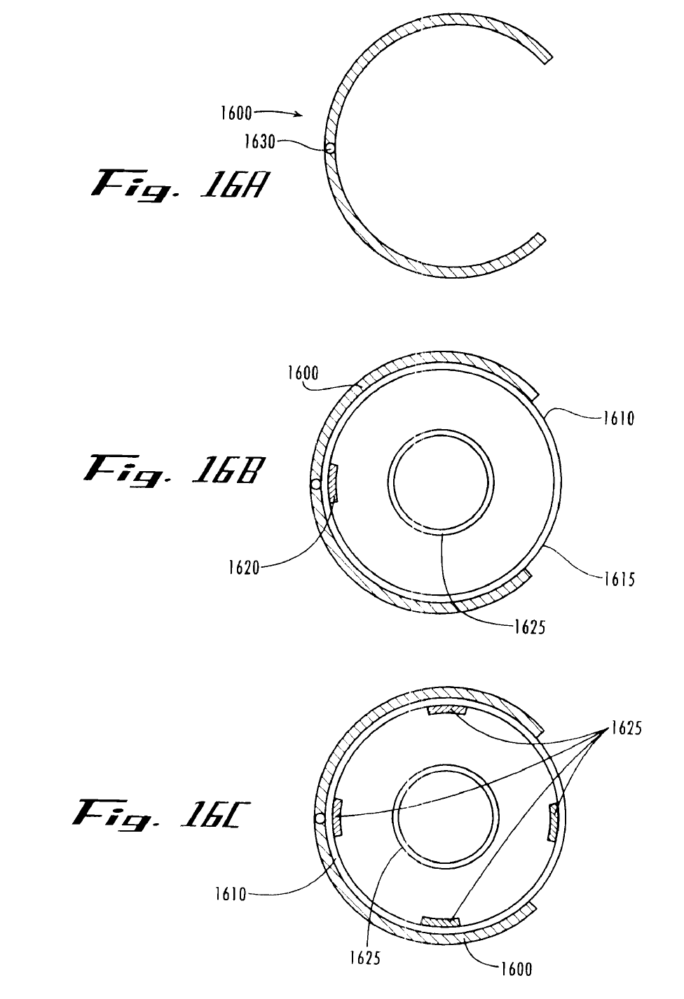

In yet another embodiment according to the present invention, FIGS. 15 A-D detail a balloon cannula 1500 inserted into a blood vessel lumen 1525. The cannula shaft 1505 is provided with an inflatable balloon 1510 contained within a recessed trough 1540 in the wall of the cannula shaft 1505, such that the inflatable balloon 1510 is substantially flush with the wall of the cannula shaft 1505 when the inflatable balloon 1510 is deflated. The wall of the balloon contains one or more intralumenal magnets 1515. When the inflatable balloon is inflated 1510, as shown in FIG. 15 C and 15 D, the intralumenal magnets 1515 are displaced to rest against the blood vessel lumen 1525. FIGS. 15 E-H show an alternative embodiment of the present invention with multiple intralumenal magnets 1515, as previously described. In various embodiments according to the present invention, intralumenal magnets may be employed with either smooth or frictional surfaces, and may be coated or uncoated (not shown).

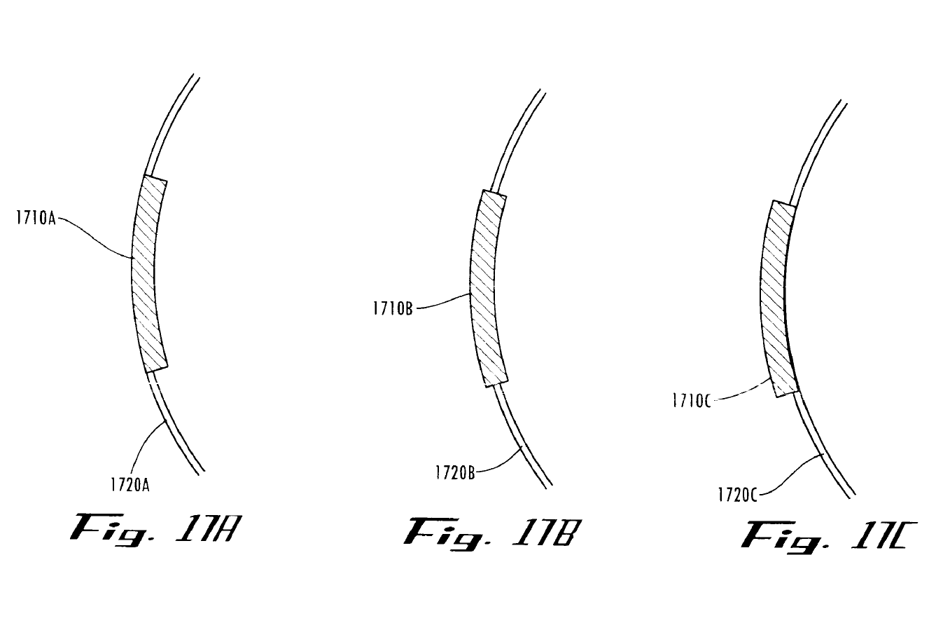

FIGS. 16 A-C show an exemplary embodiment of an extravascular magnetic collar 1600 which may be employed with the balloon cannula of FIGS. 15 A-H to magnetically stabilize the position of a magnetic intravascular balloon cannula 1605 containing one or more intralumenal magnets 1625 within a blood vessel lumen 1610. An extravascular magnetic collar 1600 comprises a collar constructed of or containing magnets or ferrous metals that may be provided in a band with an inner surface curved to accommodate a blood vessel's outer curvature. An extravascular magnetic collar 1600 may be provided as a single piece unit, or may be provided with a hinged joint 1630 as shown in FIG. 16 A. An extravascular magnetic collar 1600 may be provided to engage a balloon cannula with a single intralumenal magnet 1620 or proper magnetic polarity as shown in FIG. 16 B, or multiple such intralumenal magnets 1620 as shown in FIG. 16 C. An extravascular magnetic collar 1600 according to the present invention may be provided to encircle a substantial portion of the cross-sectional aspect of a blood vessel containing an intravascular balloon cannula, as shown in FIGS. 16 B and 16 C, or it may provide lesser encirclement of a blood vessel and only localized magnetic engagement between an extravascular magnetic collar 1600 and a magnetic intravascular balloon cannula 1605 (not shown). In various embodiments according to the present invention, extralumenal magnets may be employed with either smooth or frictional surfaces, and may be coated or uncoated (not shown).

In use, a magnetic intravascular balloon cannula 1605 is placed at a desired location within a blood vessel lumen 1610, and its balloon 1615 expanded, displacing its intralumenal magnets 1620 against the blood vessel lumen 1610. An extravascular magnetic collar 1600 according to the present invention may be employed to the exterior surface of the same blood vessel to allow magnetic attraction of the intralumenal magnets 1620 and the extravascular magnetic collar 1600 to stabilize the positioning of the cannula 1600. In such use, the extravascular magnetic collar 1600 may further be secured with sutures or other fixation means to stabilize and maintain its position during use. After such use is complete, the extravascular magnetic collar 1600 may be removed first, and then the magnetic intravascular balloon cannula 1605 may be deflated to allow its removal. In alternative embodiments according to the present invention, extravascular magnetic stabilizers (not shown) may be employed, utilizing one or more extravascular magnets deployed externally to a blood vessel without a collar as shown in FIGS. 16 A-C.

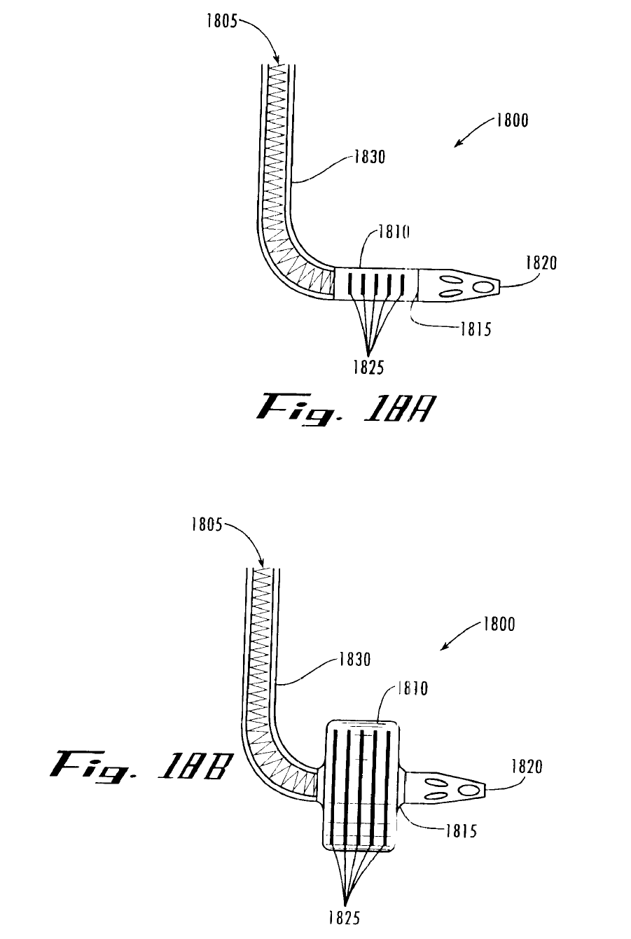

FIGS. 17 A-C show alternative configurations for the placement of intralumenal magnets 1710 with respect to the balloon wall 1720 in the magnetic intravascular balloon cannulae of FIGS. 15 A-H and 16 A-C. In FIG. 17 A, the intralumenal magnet 1710A is mounted such that its outer surface is substantially flush with the outer wall of the inflated balloon wall 1720A. In FIG. 17 B, the intralumenal magnet 1710B is mounted such that its structure is substantially centered within the thickness of the outer wall of the inflated balloon wall 1720B. In FIG. 17 C, the intralumenal magnet 1710C is mounted such that its outer surface is mounted on the outer wall of the inflated balloon wall 1720C, and does not extend significantly into the balloon lumen within the inflated balloon wall 1720C.

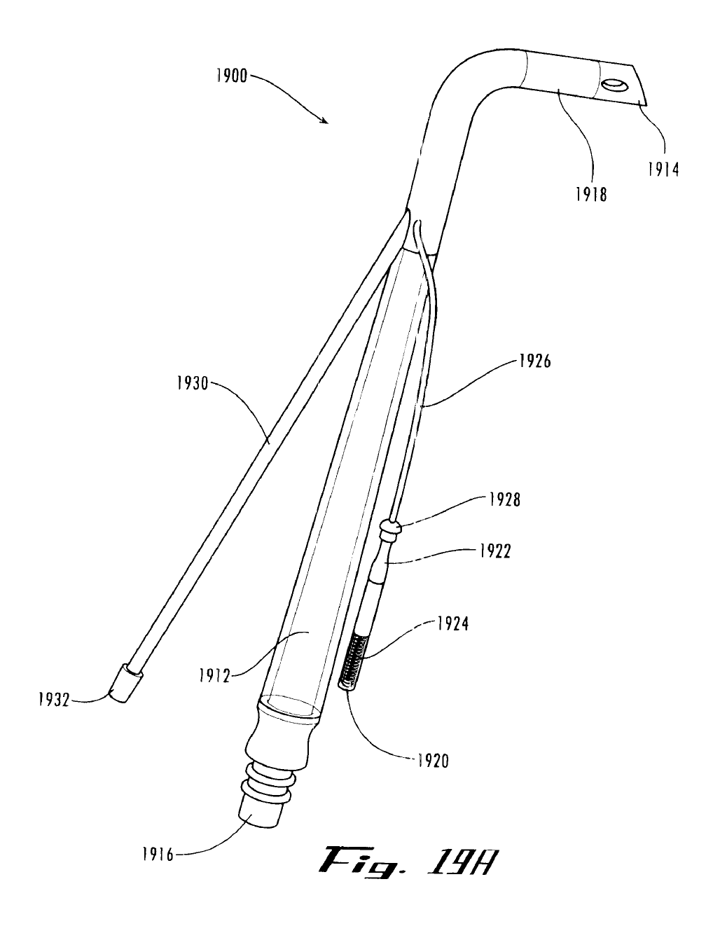

FIGS. 18 A-B detail yet another alternate embodiment according to the present invention, with a balloon cannula 1800 comprising an offset circular cannula lumen 1805 ending at a distal cannula tip 1820. Proximal to the distal cannula tip 1820, an integral inflatable balloon 1810 is substantially flushly housed in a trough 1815 in the cannula shaft 1830 of the balloon cannula 1800 when said balloon 1810 is deflated. In this embodiment according to the present invention, the integral inflatable balloon 1810 is further provided with one or more transverse retention elements 1825. Such transverse retention elements 1825 may consist of ridges or other friction-providing structures positioned to contact the intimal surface of a blood vessel and retard slippage of said balloon 1810 when deployed and inflated within said blood vessel, as shown in FIG. 18 B. Transverse retention elements 1825 may be solid, hollow, or inflatable in conjunction with inflation of the integral inflatable balloon 1810. When the integral inflatable balloon 1810 is deflated, as in FIG. 18 A, the transverse retention elements 1825 are contained within the trough 1815 of the balloon cannula 1800, such that the inflatable balloon 1810 and the transverse retention elements 1825 are substantially flush with the wall of the cannula shaft 1830 to allow for smooth and atraumatic ingress and egress from the blood vessel lumen.

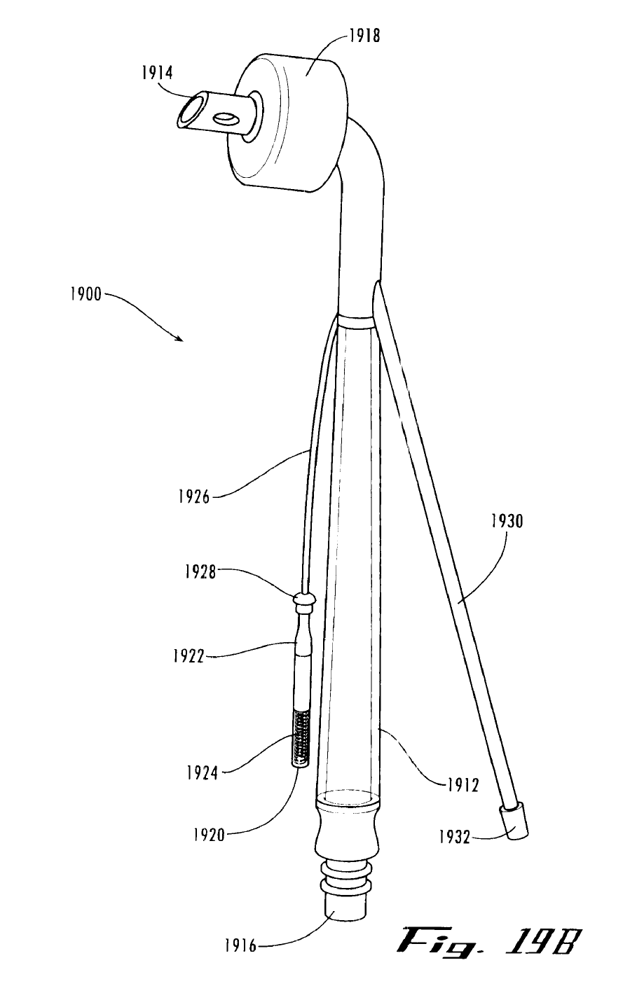

An exemplary embodiment of an arterial balloon cannula according to the present invention is detailed in FIGS. 19 A-B, where an arterial balloon cannula 1900 includes an elongated, tubular cannula shaft 1912 having a distal end 1914 and a proximal end 1916. Not shown in FIGS. 19 A-B, but similar to FIG. 6, the balloon cannula shaft 1912 contains an offset circular cannula lumen, with the offset location producing a thickened area of the cannular wall. An inflatable member or balloon 1918 is located adjacent the distal end 1914 of the cannula shaft 1912 in a trough within the cannula shaft 1912, such that the balloon 1918 is substantially flushly housed in said trough in the cannula shaft 1912 of the arterial balloon cannula 1900 when said balloon 1918 is deflated. An inflation valve 1920 adapted to accommodate a syringe or other suitable apparatus branches off from the cannula shaft 1912 at a location adjacent the proximal end 1916 of the cannula shaft. A syringe can be coupled to the inflation valve 1920 to inflate or to deflate the balloon 1918 by infusing liquids or gases into the inflatable member through an inflation lumen. In the disclosed embodiment, a pilot balloon 1922 is provided in-line between the inflation valve 1920 and the balloon 1918. The pilot balloon 1922 is inflatable and is designed to provide a user an indication of the degree of inflation of the balloon 1918. A medication delivery lumen extends parallel to the cannula lumen, utilizing space within the thickened wall of the cannular wall (not shown) so as to maintain both the circular inner cannular lumen and circular outer cannular wall. Continuous with the medication delivery lumen, and shown in FIGS. 19 A-B, is a delivery conduit 1930, terminating in a delivery connector 1932 sized and configured to connect with a syringe (not shown) or other suitable apparatus for drug delivery therethrough. FIG. 19 A shows the arterial balloon cannula 1900 with the balloon 1918 deflated. FIG. 19 B shows the arterial balloon cannula 1900 with the balloon 1918 inflated. Various other embodiments of the arterial balloon cannula 1900 of FIGS. 19 A-B may also incorporate retention and/or filtration devices not shown in FIGS. 19 A-B, but similar to those devices as disclosed elsewhere herein.

The present invention further includes methods for the cannulation of anatomic vascular structures containing blood flowing under pressure in which said cannulation can be achieved with an internal seal to prevent backflow or leakage of blood retrograde to the direction of cannula placement, with an internal cannular lumen which is circular throughout its length and maintained near the center of the vascular luminal flow, and with an inflatable balloon member which is substantially flush with the external cannular wall surface when in a deflated condition for insertion or removal. These methods according to the present invention allow for the atraumatic placement and removal of a deflated balloon cannula within a vascular lumen, while preserving the optimal flow characteristics of a truly circular internal lumen that can be maintained at or near the center of flow within the anatomic vascular lumen.

In addition, the present invention also may include methods for placing a balloon cannula within a blood vessel and maintaining its position therein to retard tendencies of an inflated balloon cannula to dysfunctionally migrate or cause injury to the endothelial surface within the blood vessel, especially under the pressure of cardiopulmonary bypass or other infusion or transfusion pump flow. In various balloon cannulae according to the present invention as illustrated herein, such methods may employ retention devices which may be inserted to retain the deployed placement of a balloon cannula, or may involve structures intrinsic to such balloon cannulae that may retain such placement, either through their direct action on the vascular tissue or in conjunction with other device components that may be deployed either intravascularly or extravascularly.

Further still, the present invention includes methods of vascular cannulation that may incorporate the deployment of devices to serve as filters, traps, or otherwise interact with the endothelium and any dislodged plaque or other potentially embolic debris or matter to prevent or reduce embolic events resulting from vascular cannulation with a balloon cannula.

Although the foregoing embodiments of the present invention have been described in some detail by way of illustration and example for purposes of clarity and understanding, it will be apparent to those skilled in the art that certain changes and modifications may be practiced within the spirit and scope of the present invention. Therefore, the description and examples presented herein should not be construed to limit the scope of the present invention, the essential features of which are set forth in the appended claims.

[image]

What is claimed is:

1. A balloon cannula for placement in a blood vessel or other vascular structure having an inner surface, comprising:
an elongated shaft of circular cross-sectional shape, said shaft having an exterior surface and having proximal and distal ends; a main lumen of circular cross-sectional shape extending substantially the entire length of said shaft;
a circumferential trough formed in said exterior surface of said elongated shaft, said circumferential trough having a reduced diameter in comparison to the adjacent circumference of said elongated shaft, and said trough having proximal and distal edges;
a balloon overlying said circumferential trough of said elongated shaft, said balloon having a wall with interior and exterior surfaces, and said wall having proximal and distal portions, said interior surface of said balloon wall being secured at said proximal and distal portions to said proximal and distal edges of said trough in a fluid-tight manner; and
an inflation lumen formed in said elongated shaft and terminating at a port opening beneath said balloon, whereby fluid or gas can be infused and withdrawn through said inflation lumen to inflate and to deflate said balloon.

2. The balloon cannula of claim 1, wherein said balloon and said trough are configured such that when said interior surface of said balloon wall is imposed against said trough in an uninflated state, said exterior surface of said balloon wall is flush with said adjacent circumference of said elongated shaft.

3. The balloon cannula of claim 1, further comprising an accessory lumen formed within said shaft and terminating in an aperture located distal to said balloon.

4. The balloon cannula of claim 1, further comprising an accessory lumen formed within said shaft and terminating in an aperture located proximal to said balloon.

5. A balloon cannula, comprising:
an elongated shaft, said elongated shaft defining a longitudinal lumen, said elongated shaft having a central longitudinal axis, and said longitudinal lumen having a central longitudinal axis laterally offset from said central longitudinal axis of said elongated shaft;
whereby said longitudinal lumen is eccentric with respect to said elongated shaft.

6. The balloon cannula of claim 5, wherein said longitudinal lumen comprises a main longitudinal lumen, wherein said longitudinal lumen being eccentric with respect to said elongated shaft defines a wall section on a first side of said longitudinal lumen having a greater thickness than a wall section on an opposite second side of said longitudinal lumen; and wherein said balloon cannula further comprises a second longitudinal lumen formed in said wall section of greater thickness.

7. The balloon cannula of claim 5, further comprising at least one accessory lumen formed within said shaft and terminating in an aperture located distal to said balloon.

8. The balloon cannula of claim 7, wherein at least one accessory lumen within said shaft is sized and configured to receive a deployable retention element to be placed therethrough.

9. The balloon cannula of claim 7, wherein at least one accessory lumen within said shaft is sized and configured to receive a deployable filtration element to be placed therethrough.

10. The balloon cannula of claim 5, further comprising at least one intralumenal magnet attached to or incorporated within said exterior surface of said balloon wall and configured such that when said interior surface of said balloon wall is imposed against said trough in an uninflated state, said exterior surface of said balloon wall including said magnet is flush with said adjacent circumference of said elongated shaft.

11. The balloon cannula of claim 10, further comprising at least one extralumenal magnet that may be position on or adjacent to a blood vessel containing said balloon cannula, such that magnetic attraction between said extralumenal and intralumenal magnets is sufficient to retard unintentional displacement of said balloon cannula within said blood vessel during use.

12. The balloon cannula of claim 5, further comprising at least one transverse retention element attached to or incorporated within said exterior surface of said balloon wall and configured such that when said interior surface of said balloon wall is imposed against said trough in an uninflated state, said exterior surface of said balloon wall including said transverse retention element is flush with said adjacent circumference of said elongated shaft, but upon inflation of said balloon, said transverse retention element is compressed against the inner surface to the blood vessel or other vascular structure to provide frictional or other mechanical stabilization sufficient to retard unintentional displacement of said balloon cannula within said blood vessel during use.

13. The balloon cannula of claim 5, further comprising an accessory lumen formed within said shaft and terminating in an aperture located proximal to said balloon.

14. The balloon cannula of claim 13, wherein at least one accessory lumen within said shaft is sized and configured to receive a deployable retention element to be placed therethrough.

15. The balloon cannula of claim 13, wherein at least one accessory lumen within said shaft is sized and configured to receive a deployable filtration element to be placed therethrough.

16. The balloon cannula of claim 13, further comprising at least one intralumenal magnet attached to or incorporated within said exterior surface of said balloon wall and configured such that when said interior surface of said balloon wall is imposed against said trough in an uninflated state, said exterior surface of said balloon wall including said magnet is flush with said adjacent circumference of said elongated shaft.

17. The balloon cannula of claim 16, further comprising at least one extralumenal magnet that may be position on or adjacent to a blood vessel containing said balloon cannula, such that magnetic attraction between said extralumenal and intralumenal magnets is sufficient to retard unintentional displacement of said balloon cannula within said blood vessel during use.

18. The balloon cannula of claim 5, further comprising at least one transverse retention element attached to or incorporated within said exterior surface of said balloon wall and configured such that when said interior surface of said balloon wall is imposed against said trough in an uninflated state, said exterior surface of said balloon wall including said transverse retention element is flush with said adjacent circumference of said elongated shaft, but upon inflation of said balloon, said transverse retention element is compressed against the inner surface to the blood vessel or other vascular structure to provide frictional or other mechanical stabilization sufficient to retard unintentional displacement of said balloon cannula within said blood vessel during use.

19. A method of cannulation of a blood vessel with a lumen, and outer surface, and an inner lining with a balloon cannula, wherein said balloon cannula comprises an elongated shaft of circular cross-sectional shape, said shaft having an exterior surface and having proximal and distal ends; a main lumen of circular cross-sectional shape extending substantially the entire length of said shaft; a circumferential trough formed in said exterior surface of said elongated shaft, said circumferential trough having a reduced diameter in comparison to the adjacent circumference of said elongated shaft, and said trough having proximal and distal edges; a balloon overlying said circumferential trough of said elongated shaft, said balloon having a wall with interior and exterior surfaces, and said wall having proximal and distal portions, said interior surface of said balloon wall being secured at said proximal and distal portions to said proximal and distal edges of said trough in a fluid-tight manner; and an inflation lumen formed in said elongated shaft and terminating at a port opening beneath said balloon, whereby fluid or gas can be infused and withdrawn through said inflation lumen to inflate and to deflate said balloon, wherein said method comprises:
placement of said balloon cannula through an opening made in the outer wall of said blood vessel;
advancement of said cannula a desired distance into said vessel lumen;
inflation of said balloon sufficient to seal blood flow around said inflated balloon within said vessel lumen;
perfusion through said balloon cannula into said vessel lumen; and
removal of said balloon cannula at a time desired by an operator by deflation of said balloon and withdrawal of said balloon cannula from said vessel lumen.

20. The method of claim 19, wherein said balloon cannula further comprises a retention element.

21. The method of claim 20, wherein at least one said retention element is deployed through an accessory lumen traversing at least a portion of the length of the shaft of said balloon cannula.

22. The method of claim 20, wherein said retention element comprises at least one intralumenal magnet when said balloon cannula is deployed for use in a blood vessel, such that said balloon cannula may be held in position by the interaction of said intralumenal magnet(s) with at least one extralumenal magnet placed by an operator.

23. The method of claim 20, wherein said retention element comprises at least one transverse retention element attached to or incorporated within said exterior surface of said balloon wall and configured such that when said interior surface of said balloon wall is imposed against said trough in an uninflated state, said exterior surface of said balloon wall including said transverse retention element is flush with said adjacent circumference of said elongated shaft, but upon inflation of said balloon, said transverse retention element is compressed against the inner surface to the blood vessel or other vascular structure to provide frictional or other mechanical stabilization sufficient to retard unintentional displacement of said balloon cannula within said blood vessel during use.

24. The method of claim 19, wherein said balloon cannula further comprises a deployable filtration element, operable to prevent or reduce the incidence of embolism during use of said balloon cannula.
